(19) United States
(12) Patent Application Publication
(10) Pub. No.: US 2021/0378747 A1
(43) Pub. Date: Dec. 9, 2021

(54) METHOD AND SYSTEM FOR COMPUTATIONAL MODELING AND SIMULATION OF THE OPERATION AND/OR BEHAVIOR OF MEDICAL DEVICES WHICH ARE IMPLANTABLE OR USABLE ON PATIENTS

(71) Applicant: INSILICOTRIALS TECHNOLOGIES S.r.l., Trieste (IT)

(72) Inventors: Luca EMILI, Trieste (IT); Alessia BARETTA, Trieste (IT); Vincenzo CARBONE, Trieste (IT); Elena LUCANO, Trieste (IT); Alberto PALAZZIN, Italy (IT)

(21) Appl. No.: 17/042,759

(22) PCT Filed: Mar. 26, 2019
(86) PCT No.: PCT/IB2019/052437

§ 371 Date: Sep. 28, 2020

(30) Foreign Application Priority Data
Mar. 28, 2018 (IT) 102018000004044

(51) Int. Cl. A61B 34/10; A61B 34/00; G06F 30/20

(52) U.S. Cl. CPC A61B 34/10; G06F 2111/16; G06F 30/20; A61B 34/25

(57) ABSTRACT

A method for a functional and/or structural computational modeling and simulation of medical devices which are implantable or usable on patients is described.

The method first comprises the steps of storing on a computer platform 1 digital modeling data D1 of medical device, adapted to model the function and/or structure and/or behavior of a medical device which is implantable or usable on a patient, referred to the whole medical device or to a part thereof; and storing in the computer platform 1 digital anatomical and/or physiological modeling data D2 of a real or virtual patient, referred to one or more anatomical parts of the patient with which the medical device is intended to interact.

The method then includes providing, through the computer platform 1, a user interface 4 which can be connected to the Internet and configured to allow a user to connect and interact with the computer platform 1 of digital data and with one or more software programs included therein.

The method then comprises the step of receiving selection and/or setting information I which can be entered by the user through the user interface 4. Such selection and/or setting information comprises: information (I1) of selection and/or definition and/or setting of a medical device model; information (I2) of selection and/or definition and/or setting of an anatomical and/or physiological model of patient based on said stored anatomical and/or physiological modeling digital data; information (I3) of selection and setting of a simulation type, and/or information of selection and setting of one or more input simulation parameters (I4) and/or one or more output simulation parameters (I5).

The method then includes processing, by means of the computer platform 1, the aforementioned information for the selection and/or definition and/or setting of a medical device model 11 to prepare a model M1 of medical device based on the aforementioned modeled digital data D1 for the functional and/or structural and/or modeling of behavior of the medical device; furthermore, by means of the computer platform 1, processing the aforementioned information for the selection and/or definition and/or setting of a patient model I2 to prepare an anatomical and/or physiological model M2 of one or more anatomical parts of the patient based on the stored anatomical and/or physiological modeling digital data D2; furthermore, by means of the computer platform, processing the aforementioned selection and/or setting information I entered by the user to prepare input setting data Din (I3, I4) for one or more computational simulation software programs included in the computer platform.

A step of executing the computational simulation is then provided, by the one or more computational simulation software programs, on the basis of said input setting data Din, of said medical device model M1 and of said anatomical and/or physiological model M2.

Finally, the method comprises the steps of processing the data Dout obtained from the computational simulation, on the basis of the information I5 on the selection and setting of one or more simulation output parameters, to express the desired simulation results R in a format selected by the user; and providing the desired simulation results through the user interface 4.

### TECHNICAL BACKGROUND OF THE INVENTION

### Field of Application

The present invention generally relates to the technical field of computational modeling and simulation by means of electronic processors in the area commonly defined as “in silico clinical trials” or more simply “in silico trials”.

In particular, the invention relates to a method and system for the functional and/or structural computational simulation and modeling of medical devices which are implantable or usable on patients.

In other words, the invention relates to a method and system for computational modeling and simulation of the operation and/or behavior of medical devices which are implantable or usable on patients.

### Description of the Prior Art

The technical field of “in silico trials” relates to computational modeling and simulations carried out to support the development, testing, evaluation or certification of medical or clinical devices. The use of “in silico trials” increasingly emerges as advantageous to complement, complete or partially replace the so-called “in vivo clinical trials”, i.e. experimental evaluations of medical devices performed on real patients.

For this reason, different types of computational simulation software programs or packages have been developed and are known, even technically very advanced, being capable of simulating the structure and behavior of a medical device, for example.

Typically, the computational simulation programs or software packages known in this area are very complex and expensive. Their use also requires combining specific medical and IT skills (for example, to control the setting of numerous simulation parameters, many of which are related to clinical aspects).

In the more specific area of “in silico trials” concerning devices which are implantable or usable on patients, even more specific and stringent needs emerge.

First, the importance of “in silico trials” becomes even more important, and sometimes necessary, to carry out simulation tests of the device behavior once implanted or used on the patient (for which the equivalent experimental tests could not be carried out before the implant itself).

Moreover, in this clinical context, particularly reliable computational simulations are required, which are able to provide results not only on structural behavior, but also on the operation. For this reason, the need to take into account in some way the anatomy and the functionality of the parts of the body with which the devices are intended to interact also emerges (which is obtained, in some known solutions, through a very accurate setting of appropriate boundary conditions of some simulation parameters). This further increases the complexity and difficulty of using computational simulation programs or software packages.

In summary, in the technical field of “in silico trials” concerning devices which are implantable or usable on patients, there is a particular need to have systems and methods of computational modeling and simulation which are able to simulate the medical device both structurally and functionally, taking into account as precisely as possible the features of the device and the patient and their interaction, thus providing results of sufficient accuracy for use in tests, evaluations, certifications, and so on.

At the same time, the need arises for these systems and methods of computational modeling and simulation to be as simple, available and easily accessible as possible to all the potential users (and not only to large entities such as big clinical institutions or big medical companies) and adaptable to most of possible cases.

Currently, the known computational simulation software packages or programs do not fully meet all the above-mentioned requirements needed to perform “in silico trials” on implantable or usable devices.

### SUMMARY OF THE INVENTION

In light of the foregoing, it is the object of the present invention to provide a method of functional and/or structural computational modeling and simulation of medical devices which are implantable and/or usable on patients, which allows to obviate at least partially the drawbacks mentioned above with reference to the prior art, and meeting the aforementioned requirements particularly felt in the technical field considered.

Such an object is achieved by a method according to claim 25.

Further embodiments of such a method are defined by claims 26-41.

The present invention also relates to a system for the functional and/or structural computational modeling and simulation of medical devices which are implantable and/or usable on patients. This system is defined in claim 42.

Further embodiments of the system are defined in claims 43 and 44.

### BRIEF DESCRIPTION OF THE DRAWINGS

Further features and advantages of such a system and method according to the invention will become apparent from the following description of preferred exemplary embodiment, given by way of a non-limiting example with reference to the accompanying drawing, in which:

FIG. 1 shows a simplified block diagram of a system for the functional and/or structural computational modeling and simulation of medical devices, according to an embodiment of the present invention.

### DETAILED DESCRIPTION

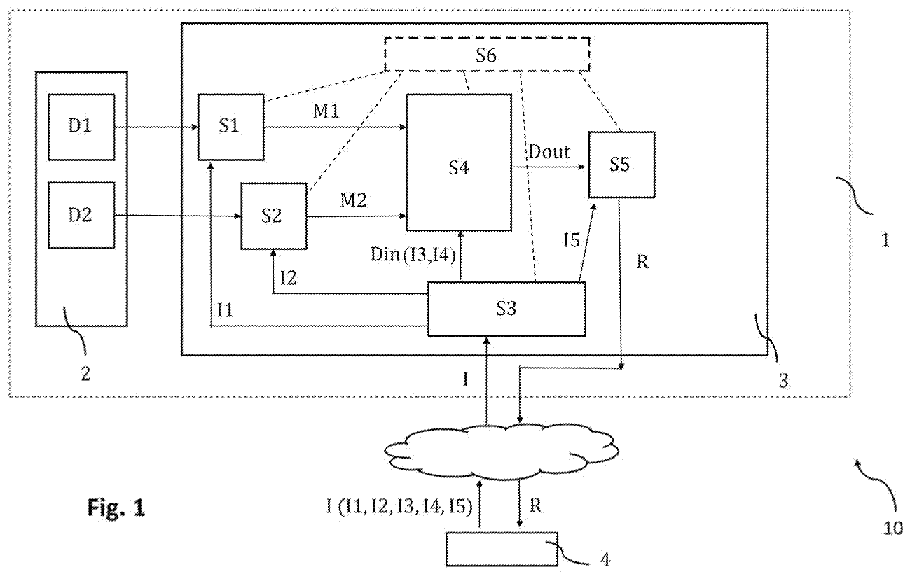

With reference to FIG. 1, a method for a functional and/or structural computational modeling and simulation of medical devices which are implantable or usable on patients is described.

The method first comprises the steps of storing on a computer platform 1 digital modeling data D1 of medical device, referred to a medical device which is implantable or usable on a patient, referred to the whole medical device or to a part thereof; and storing on the computer platform 1 digital anatomical and/or physiological modeling data D2 of a real or virtual patient, referred to one or more anatomical parts of the patient with which the medical device is intended to interact.

The method then includes providing, by means of the computer platform 1, a user interface 4 which can be connected to the Internet and configured to allow a user to connect and interact with the computer platform 1 of digital data and with one or more software programs included therein.

The method then comprises the step of receiving selection and/or setting information I which can be entered by the user through the user interface 4. Such selection and/or setting information comprises: information (I1) on the selection and/or definition and/or setting of a medical device model; information (I2) on the selection and/or definition and/or setting of an anatomical and/or physiological model of patient based on said stored anatomical and/or physiological modeling digital data; information (I3) on the selection and/or setting of a simulation type, and/or information on the selection and setting of one or more input simulation parameters (I4) and one or more output simulation parameters (I5).

The method then includes processing, by means of the computer platform 1, the aforementioned information I1 on the selection and/or definition and/or setting of a medical device model for preparing a medical device model M1 based on the aforementioned stored digital data D1 of medical device modeling; furthermore, by means of the computer platform 1, processing the aforementioned information I2 on the selection and/or definition and/or setting of a patient model for preparing an anatomical and/or physiological model M2 of one or more anatomical parts of the patient based on the stored anatomical and/or physiological modeling digital data D2; furthermore, by means of the computer platform, processing the aforementioned selection and/or setting information I entered by the user to prepare input setting data Din (in particular dependent on I3, I4) for one or more computational simulation software programs included in the computer platform.

A step of executing the computational simulation is then provided, by the one or more computational simulation software programs, on the basis of said input setting data Din, of the aforementioned medical device model M1 and of the aforementioned anatomical and/or physiological model M2, to obtain output data Dout of the computational simulation.

Finally, the method comprises the steps of processing the output data Dout of the computational simulation, on the basis of the information I5 on the selection and setting of one or more simulation output parameters, to express simulation results R, representative of a functional and/or structural behavior of the medical device and/or patient (i.e., the results desired by the user), in a format selected by the user; and of providing said simulation results through the user interface 4.

According to an embodiment, the method is adapted to perform a computational modeling and simulation of the operation and/or behavior of medical devices which are implantable and/or usable on patients. The medical device digital modeling data D1 are adapted to model the function and/or structure and/or behavior of the medical device which is implantable or usable on a patient, referred to the entire medical device or a part thereof.

According to an embodiment of the method, the step of providing a user interface comprises providing a plurality of user-selectable templates associated with respective types of simulation. Each of these templates includes: a plurality of input parameters which can be selected for the simulation, in which each parameter is associated with a respective range of allowed values (for example, values appropriate for the feasibility of the simulation), within which a value of the parameter can be set; a plurality of selectable output parameters, comprising the desired quantities as the output result; a plurality of presentation and reporting options, which can be selected by the user to choose the format of the results and/or the methods to analyze the results themselves.

In this embodiment, the templates can be predefined in many diverse ways, with regard to the choice of the simulation input and output parameters and the relative allowed intervals, and regarding the presentation and reporting of the results, permitting much easier management of the simulation by the user.

According to different possible embodiments of the method, the selection and/or setting information I which can be entered by the user also includes parameters for the definition and/or specialization of the medical device digital model Ml; and/or parameters for defining and/or customizing the anatomical and/or physiological digital model M2 of one or more real or virtual patients; and/or parameters for the selection and/or definition of real or virtual patients, or real or virtual patient populations; and/or parameters for setting computational aspects of the simulation.

According to various possible implementation options, the selection and/or setting information (I) which can be entered by the user includes initial conditions and boundary conditions for the simulation, and/or geometric parameters and/or parameters related to the properties of the medical device materials, and/or parameters related to the numerical method used by the computational simulation software.

According to an embodiment, the method further comprises the steps of obtaining digital modeling data D1 of a medical device by selecting from a plurality of digital models of medical devices stored in a digital library 2 of the computer platform 1 and/or pre-loaded by the user on the computer platform; and furthermore obtaining digital modeling data D2 of one or more patients selecting from a plurality of digital models of real or virtual patients stored in the digital library 2 of the computer platform 1 and/or pre-loaded by the user on the computer platform.

The aforementioned “digital device model” and “digital patient model” may include, for example, structured digital data sets designed to digitally represent a device or patient, or respective parts, or respective properties; these structured digital data sets are capable of being processed or processed by computational simulation programs or software packages.

In a particular embodiment of the method, the digital modeling data of a real patient are anonymized and/or de-identified and/or pseudonymized.

According to an implementation option, the digital modeling data of a virtual patient is obtained by statistical processing of digital modeling data of several real patients.

According to different possible implementation options of the method, the computational simulation includes, among others, structural simulations and/or fluid-dynamics simulations and/or thermal simulations and/or electromagnetic simulations and/or biomechanical simulations.

The method, in itself, can be applied in conjunction with a vast plurality of computational simulations (belonging to the aforementioned categories), characterized by the most diverse complexities, procedures, number of iterations, number and type of computational simulation programs, and so on. These computational simulations, as already observed, may concern structural and/or functional aspects of the medical device, and/or of the functional response of the patient in which the device is implanted.

Below, some significant examples of computational simulations included in the method will be explicitly indicated.

According to various implementation examples of the method, computational simulations include simulations for the design and/or development of the medical device, and/or for analysis and prediction of the behavior of the medical device on a population of real or virtual patients, and/or for a customized assessment of the effects of the medical device on a specific real or virtual patient, and/or for evaluations of safety and/or efficacy and/or compliance of the medical device with current regulations.

According to an example of application of the method, the medical device is a prosthesis (for example, an orthopaedic prosthesis), and the prosthesis model comprises a three-dimensional structural model, the anatomical model of the patient comprises a three-dimensional physiological model and the simulation comprises structural simulations of the mechanical behavior of the implanted medical device and/or the patient's biomechanical response to the implanted device.

According to another application example of the method, the medical device is an endovascular device, the model of the endovascular device comprises a fluid-dynamic model, and the simulation comprises a fluid-dynamic simulation of the blood flow.

According to a further application example of the method, the medical device is a device which is implantable in the patient's body, the model of the implantable device comprises a behavioral and/or thermal model under electromagnetic field effect, and the simulation comprises an electromagnetic simulation adapted to simulate the effect of the electromagnetic field on the temperature variation of the patient's tissues and/or the behavior of the device.

According to another application example of the method, the medical device is an implantable drug-releasing device, the model of the implantable device comprises a fluid-dynamic model, the anatomical and/or physiological model comprises a parameter related to permeability, and the simulation comprises fluid-dynamic and/or functional simulations.

With reference again to FIG. 1, a system for a functional and/or structural computational modeling and simulation of medical devices which are implantable and/or usable on patients comprising a computer platform 1 is described.

This computer platform comprises at least one digital library 2 and one or more electronic processing components 3 (i.e., electronic processors) in which one or more software programs or applications (S1-S6) are stored and can be executed.

In the digital library 2, digital modeling data D1 of a plurality of medical devices implantable or usable on a patient are stored, referring to the medical device, or to a part thereof (such digital data D1 being adapted to model, for example, the function and/or structure and/or behavior of the medical device or part thereof); and digital data D2 for anatomical and/or physiological modeling of a plurality of real or virtual patients are also stored, referring to one or more anatomical parts of the patient with which the medical devices are intended to interact.

The aforesaid one or more processing components 3 are configured to perform, by means of the one or more software programs or applications (S1-S6) stored and executed therein, the actions of: providing a user interface 4 which can be connected to the Internet and configured to allow a user to connect and interact with the digital data processing platform 1 and with one or more software programs included therein; then, receiving selection and/or setting information I which can be entered by the user through the user interface 4. Such selection and/or setting information comprises: information (I1) on the selection and/or definition and/or setting of a medical device model; information (I2) on the selection and/or definition and/or setting of an anatomical and/or physiological model of patient based on said stored anatomical and physiological modeling digital data; information (I3) on the selection and/or setting of a simulation type, and/or information on the selection and/or setting of one or more simulation input parameters (I4) and one or more simulation output parameters (I5).

The one or more processing components 3 are further configured to carry out, by means of one or more software programs or applications (S1-S6), the following further actions: processing the aforementioned information I1 on the selection and/or definition and/or setting of a medical device model for preparing a medical device model M1 based on the stored digital data D1 of functional and/or structural modeling and/or behavior of medical device; furthermore, processing the aforementioned information I2 on the selection and/or definition and/or setting of a patient model I2 for preparing an anatomical and/or physiological model M2 of one or more anatomical parts of the patient based on the stored anatomical and/or physiological modeling digital data D2; furthermore, processing the aforementioned selection and/or setting information I entered by the user to prepare input setting data Din (in particular dependent on I3, I4) for one or more computational simulation software programs S4 included in the computer platform 1; then, carrying out the computational simulation, by the one or more computational simulation software programs S4, based on the input setting data Din, on the medical device model M1 and on the anatomical and/or physiological model M2, to obtain output data Dout of the computational simulation; finally, processing the output data Dout of the computational simulation, based on the aforementioned information I5 on the selection and setting of one or more output parameters of the simulation, to express simulation results R, representative of a functional and/or structural behavior of the patient and/or medical device (i.e., for example, the results desired by the user), in a format selected by the user; and providing the aforementioned simulation results R through the user interface 4.

According to an embodiment of the system 10, the aforementioned one or more software programs or applications (S1-S6) of the computer platform comprise: one or more user interface management software programs S3; one or more computational simulation software programs S4, configured to perform computational simulation when executed by an electronic processor; one or more software processing programs (S1, S2, S3, S5), configured to perform the steps of processing the information on the selection and/or definition and/or setting of a medical device model, processing the information on the selection and/or definition and/or setting of a patient model, and processing the selection and/or setting information entered by the user for preparing input setting data for the one or more computational simulation software programs and processing the output data (Dout) of the computational simulation to express the desired simulation results (R).

According to an implementation option, the computer platform 1 further comprises a PIDO (Process Integration and Design Optimization) software program S6, configured to manage the workflow of the software programs comprised in the computer platform and to optimize the computational simulations.

With reference to the aforementioned software programs included in the system, the most various options, per se known, for the implementation, partition, storage, execution of such software programs, as individual programs or as packages of programs and/or software modules interacting with each other, may be contemplated.

Several options are possible for the practical implementation of the system, from an infrastructural point of view. Among these, concentrated or distributed platforms, based on one or more interacting servers and/or computers, may be contemplated.

For example, the system can be implemented using a distributed cloud computing platform.

According to further implementation examples, the system is configured to execute a method according to any one of the embodiments of the method described above.

The above description of different embodiments of the system and of the method allows the invention to be implemented by those skilled in the art.

By way of example, further implementation details are provided below with reference to the particular example of electromagnetic simulation designed to simulate the effect of the electromagnetic field generated by a magnetic resonance (MR) system on the temperature variation of the patient's tissues due to the presence of a medical device. In this case, the medical device can be a stent modeled using a 3D model; the patient model can be a 3D ASTM phantom structural model according to the indications of the ASTM F2182 test standard. The setting parameters of the medical device model which can be set by the user (I1) may include structural features of the 3D model of stents such as length, diameter, material. The setting parameters of the patient model which can be set by the user (I2) may include the position of the ASTM phantom within the MR system. The setting parameters of a type of simulation which can be selected by the user (I3) may include setting parameters for an electromagnetic and thermal simulation. The input parameters for the simulation which can be set by the user (I4) may include features of the electromagnetic field to which the patient model with an implanted stent (e.g. frequency, power, polarization) is exposed and duration of the MR examination for the definition of increase in the patient tissue temperature. The output parameters which can be set by the user (I5) may include the spatial resolution to which the distribution of the specific absorption rate and electromagnetic field is to be represented. The results (R) of the simulations may include value and location of the maximum of the specific absorption rate and temperature found within the patient model due to the exposure to the electromagnetic field.

The software program (S1) for processing the selection and/or definition information and/or setting of a medical device model may include a “computer assisted design” (CAD) software to adapt the parameterized geometry of the stent to the parameters entered by the user. The software program (S2) for processing the selection and/or definition and/or setting information of a patient model can include CAD software to adapt the positioning of the phantom within the MRI system. The user interface management software program (S3) may include a content management system (CMS) for websites. The computational simulation software program (S4) may include finite element software for electromagnetic and thermal analysis. The software program (S5) for processing data obtained from computational simulation may include the programming language code for processing the results of electromagnetic field specific absorption rate and temperature. The PIDO software program (S6) can include a programming language code to manage the workflow of electromagnetic and thermal simulations.

The very detailed example, given above with reference to an electromagnetic simulation, can also be applied by those skilled in the art, mutatis mutandis, to the other types of simulation previously mentioned.

As can be seen, the object of the present invention is fully achieved by the method and by the system described above, by virtue of their functional and structural features.

In fact, due to the ability to select and/or create both a medical device model and an anatomical patient model, and to treat them in an integrated manner, the computational simulation method of the present invention is able to simulate both structurally and functionally the medical device and the functional response of the virtual patient to the implanted device, taking into account the anatomical features of the patient as precisely as possible, thus providing results of sufficient accuracy for use in tests, assessments, certifications, and so on.

Moreover, due to the processing carried out by the computer platform and the user interface provided (as previously described in detail), this computational simulation method allows a simplified use, and is easily accessible to anyone (suitably authorized) having access to the platform. The user interface provides the broadest possibilities for customization and adaptation of the simulation, while guiding and facilitating the setting of the simulation.

The features mentioned above, in turn, allow the creation of a shared and collaborative work environment between different entities which can access the platform, both as data providers and as entities/users requesting a simulation service, making the methodology effective also in its application aspects.

Similar advantages can be identified with reference to the system, described above, capable of executing the method of the invention.

In order to meet incidental needs, those skilled in the art may make several changes, adjustments and adaptations to the embodiments of the system and method according to the invention, and may replace elements with others which are functionally equivalent, without departing from the scope of the following claims. Each of the features described as belonging to a possible embodiment can be achieved irrespective of the other embodiments described.

1-24. (canceled)

25. A method for functional and/or structural computational modeling and simulation of medical devices which are implantable and/or usable on patients, comprising the steps of:
storing on a computer platform digital modeling data of medical device, referred to a medical device which is implantable or usable on a patient, referred to the whole medical device or a part thereof;
storing on the computer platform anatomical and/or physiological digital modeling data of a real or virtual patient, referred to one or more anatomical parts of the patient with which the medical device is intended to interact;
providing, by means of the computer platform, a user interface which can be connected to the Internet and configured to allow a user to connect and interact with the computer platform of digital data and with one or more software programs included therein;
receiving selection and/or setting information which can be entered by the user through the user interface, wherein the selection and/or setting information comprises:
information on the selection and/or definition and/or setting of a medical device model;
information on the selection and/or definition and/or setting of an anatomical and/or physiological model of a patient based on said stored digital data of anatomical and/or physiological modeling;
information on the selection and setting of a simulation type, and/or information on the selection and setting of one or more input simulation parameters, and one or more output simulation parameters;
processing, by means of the computer platform, said information on the selection and/or definition and/or setting of a medical device model for preparing a medical device model based on said stored digital data of medical device modeling;
processing, by means of the computer platform, said information on the selection and/or definition and/or setting of a patient model for preparing an anatomical and/or physiological model of one or more anatomical parts of the patient based on said stored digital data of anatomical and/or physiological modeling;
processing, by means of the computer platform, said selection and/or setting information entered by the user for preparing input setting data for one or more computational simulation software programs included in the computer platform;
executing the computational simulation, by the one or more computational simulation software programs, on the basis of said input setting data, of said medical device model and of said anatomical and/or physiological model, to obtain output data of the computational simulation;
processing the output data of the computational simulation on the basis of said information on the selection and setting of one or more output simulation parameters, to express simulation results, representative of a functional and/or structural behavior of the medical device and/or patient, in a format selected by the user;
providing the simulation results by means of the user interface.

26. A method according to claim 25, wherein the method is adapted to perform a computational modeling and simulation of the operation and/or behavior of medical devices which are implantable and/or usable on patients,
and wherein the digital modeling data of medical device are adapted to model the function and/or structure and/or behavior of the medical device which is implantable or usable on a patient, referred to the whole medical device or a part thereof.

27. A method according to claim 25, wherein the step of providing a user interface comprises providing a plurality of user-selectable templates, associated with respective types of simulation, and wherein each template comprises:
a plurality of input parameters which can be selected for the simulation, each parameter being associated with a respective range of permitted values, within which a parameter value can be set;
a plurality of selectable output parameters, comprising the desired quantities as an output result;
a plurality of displaying and reporting options, which can be selected by the user to choose the format of the results and/or the methods to analyze the results.

28. A method according to claim 25, wherein the selection and/or setting information which can be entered by the user further comprises parameters for the definition and/or specialization of the digital model of the medical device,
and/or wherein the selection and/or setting information which can be entered by the user further comprises parameters for defining and/or customizing the anatomical and/or physiological digital model of one or more real or virtual patients.

29. A method according to claim 25, wherein the selection and/or setting information which can be entered by the user further comprises parameters for selecting and/or defining real or virtual patients, or populations of real or virtual patients.

30. A method according to claim 28, wherein the selection and/or setting information which can be entered by the user further includes geometric parameters and/or parameters related to the properties of the medical device materials.

31. A method according to claim 25, wherein the selection and/or setting information which can be entered by the user comprises parameters for setting computational aspects of the simulation,
and/or initial conditions and boundary conditions for the simulation,
and/or parameters related to the numerical method used by the computational simulation software.

32. A method according to claim 25, further comprising the steps of:
obtaining digital modeling data of a medical device by selecting from a plurality of digital models of medical devices stored in a digital library of the computer platform and/or pre-loaded by the user on the computer platform;
obtaining digital modeling data of one or more patients by selecting from a plurality of digital models of real or virtual patients stored in the digital library of the computer platform and/or pre-loaded by the user on the computer platform.

33. A method according to claim 25, wherein the digital modeling data of a real patient are anonymized and/or de-identified and/or pseudonymized.

34. A method according to claim 25, wherein the computational simulation comprises structural and/or fluid-dynamic and/or thermal and/or electromagnetic and/or biomechanical simulations.

35. A method according to claim 25, wherein the computational simulations comprise simulations for the design and/or development of the medical device.

36. A method according to claim 25, wherein the computational simulations comprise simulations for the analysis and prediction of the behavior of the medical device on a population of real or virtual patients,
and/or wherein the computational simulations comprise simulations for a customized evaluation of the effects of the medical device on a specific real or virtual patient.

37. A method according to claim 25, wherein the computational simulations comprise simulations for evaluations of the safety and/or efficacy and/or compliance of the medical device with current regulations.

38. A method according to claim 25, wherein the medical device is an orthopaedic prosthesis, the orthopaedic prosthesis model comprises a three-dimensional structural model, the anatomical model of the patient comprises a three-dimensional physiological model and the simulation comprises structural simulations of the mechanical behavior of the implanted medical device and/or of the biomechanical response of the patient to the implanted device.

39. A method according to claim 25, wherein the medical device is an endovascular device, the model of the endovascular device comprises a fluid-dynamic model, and the simulation comprises a fluid-dynamic simulation of the blood flow.

40. A method according to claim 25, wherein the medical device is a device which can be implanted in the patient's body, the model of the implantable device comprises a behavioral and/or thermal model under electromagnetic field effect, and the simulation comprises an electromagnetic simulation adapted to simulate the effect of the electromagnetic field on the temperature variation of the patient's tissues and/or on the behavior of the device.

41. A method according to claim 25, wherein the medical device is an implantable drug-releasing device, the model of the implantable device comprises a fluid-dynamic model, the anatomical and/or physiological model comprises a parameter related to permeability, and the simulation comprises fluid-dynamic and/or functional simulations.

42. A system for functional and/or structural computational modeling and simulation of medical devices which are implantable and/or usable on patients comprising a computer platform, wherein the computer platform comprises:
a digital library in which the following is stored:
digital modeling data of a plurality of medical devices which are implantable or usable on a patient, referred to the medical device, or to a part thereof;
anatomical and/or physiological digital modeling data of a plurality of real or virtual patients, referred to one or more anatomical parts of the patient with which the medical devices are intended to interact;
one or more electronic processing components configured to perform, by means of one or more software programs or applications stored and executed therein, the actions of:
providing a user interface which can be connected to the Internet and configured to allow a user to connect to and interact with the computer platform of digital data and with one or more software programs included therein;
receiving selection and/or setting information which can be entered by the user by means of the user interface, wherein the selection and/or setting information comprises: information on the selection and/or definition and/or setting of a medical device model; information on the selection and/or definition and/or setting of an anatomical and/or physiological model of a patient based on said stored anatomical and/or physiological modeling digital data; information on the selection and setting of a simulation type, and/or information on the selection and setting of one or more input simulation parameters, and of one or more output simulation parameters;
processing said information on the selection and/or definition and/or setting of a medical device model for preparing a medical device model based on said stored digital data of functional and/or structural modeling and/or of modeling of the behavior of a medical device;
processing said information on the selection and/or definition and/or setting of a patient model for preparing an anatomical and/or physiological model of one or more anatomical parts of the patient based on said stored digital data of anatomical and/or physiological modeling;
processing said selection and/or setting information entered by the user for preparing input setting data for one or more computational simulation software programs included in the computer platform;
executing the computational simulation, by the one or more computational simulation software programs, on the basis of said input setting data, of said medical device model and of said anatomical and/or physiological model, to obtain output data of the computational simulation;
processing the output data of the computational simulation, on the basis of said information on the selection and setting of one or more output simulation parameters, to express simulation results, representative of a functional and/or structural behavior of the medical device and/or patient, in a format selected by the user;
providing the simulation results by means of the user interface.

43. A system according to claim 42, wherein said one or more software programs or applications of the computer platform comprise:
one or more user interface management software programs;
one or more computational simulation software programs, configured to perform the computational simulation when executed by a computer;
one or more software processing programs, configured to perform said steps of processing the information on the selection and/or definition and/or setting of a medical device model, processing the information on the selection and/or definition and/or setting of a patient model, processing the selection and/or setting information entered by the user for preparing input setting data for the one or more computational simulation software programs, and processing the output data of the computational simulation to express the desired simulation results.

44. A system according to claim 42, wherein the computer platform further comprises a PIDO (Process Integration and Design Optimization) software program, configured to manage the workflow of the software programs included in the computer platform and to optimize computational simulations,
and/or wherein the system is implemented by means of a distributed cloud computing platform.
